(19) United States
(12) Patent Application Publication
(10) Pub. No.: US 2021/0259684 A1
(43) Pub. Date: Aug. 26, 2021

(54) DEGRADABLE SURGICAL STAPLES AND METHODS OF USING SAME

(71) Applicant: Covidien LP, Mansfield, MA (US)

(72) Inventors: Gerald N. Hodgkinson, Killingworth, CT (US); Stanislaw Z. Marczyk, Stratford, CT (US); Andrew M. Miesse, Westbrook, CT (US); Robert H. Knapp, Middlebury, CT (US)

(21) Appl. No.: 17/167,284

(22) Filed: Feb. 4, 2021

Related U.S. Application Data
(60) Provisional application No. 62/979,659, filed on Feb. 21, 2020.

(51) Int. Cl. A61B 17/064; A61L 31/02

(52) U.S. Cl. CPC A61B 17/0644; A61B 2017/00004; A61L 31/028; A61L 31/022

(57) ABSTRACT

A surgical stapler deploys staples made of selectively degradable materials. Tissue resected with a stapler of the present disclosure, including staples made of the selectively degradable materials, may be subjected to treatment in the lab to degrade those materials forming the staples. In this way, a pathologist or similar hospital personnel may rapidly degrade the staples, thereby permitting testing of the entire resected tissue sample for the presence of disease.

### CROSS REFERENCE TO RELATED APPLICATION

The present application claims the benefit of and priority to U.S. Provisional Application Ser. No. 62/979,659, filed on Feb. 21, 2020, the entire disclosure of which is incorporated by reference herein.

### FIELD

This disclosure is directed to a method of using selectively degradable staples to enhance access to resected tissue specimens by pathologists, in order to improve analysis of the tissue specimens.

### BACKGROUND

When a surgical stapling and dissecting device is operated to remove tissue from a patient, a knife of the device is advanced to dissect the tissue along a cut line. This creates a wound that is sealed with rows of staples that are ejected from the device and formed through the tissue along the cut line as the tissue is dissected. The resected tissues from surgery then go to the histopathology department of the hospital to be analyzed for disease state. Most tissue specimens are easy to work with, except for stapled tissues. During the histopathological process, the tissue must be frozen using a cryostat and sliced into sections with a microtome. Unfortunately, the blade in the microtome is not capable of cutting through staples. In most cases the pathologist will cut off the stapled tissue and throw it away, which results in a direct loss of functional tissue for the patient. If disease, for example cancer, is found in the remaining tissue of the specimen, the pathologist will ask the surgeon to remove more tissue from the patient, further reducing the patient's remaining functional tissue. In other cases, the pathologist will remove the staples one by one, which could take as long as 10 minutes and then requires an additional 20 minutes of pathological preparation of the new tissue sample. This will usually leave the remaining tissue mangled and unusable, and is a primary reason why most pathologists do not attempt to remove staples.

### SUMMARY

One aspect of the disclosure is directed to methods for using selectively degradable staples in removing diseased tissue from a patient. The staples of the present disclosure are degradable in vivo, or undergo minimal degradation in vivo, but may be subjected to in vitro treatments by a pathologist or similar hospital personnel to accelerate their degradation and permit testing of resected tissue without requiring manual removal of staples or discarding of tissue that included the staples.

In aspects, a method of the disclosure includes introducing to a patient a surgical stapler, the stapler including staples formed of a degradable material, using the stapler to staple tissue adjacent an undesired portion of an organ and cutting the tissue to obtain a resected tissue sample. The resected tissue sample is removed from the patient, and the staples are contacted with a solution to degrade the degradable material forming the staples.

In some aspects, the method further includes testing the resected tissue sample to determine the presence of a disease in the resected tissue sample.

In aspects, testing the resected tissue sample is conducted to determine the presence of cancer in the resected tissue sample.

In some aspects, the staples are formed of magnesium or zinc.

In other aspects, the staples are formed of a magnesium alloy or a zinc alloy.

In aspects, the solution to degrade the degradable material forming the staples includes an acid. The acid is selected from hydrochloric acid, sulfuric acid, acetic acid, nitric acid, or combinations thereof.

In other aspects, the solution to degrade the degradable material forming the staples includes a chloride salt.

In yet other aspects, a method of the disclosure includes introducing to a patient a surgical stapler, the stapler including staples formed of a degradable material, using the stapler to staple tissue adjacent an undesired portion of an organ and cutting the tissue to obtain a resected tissue sample. The resected tissue sample is removed from the patient, the staples are contacted with a solution to degrade the degradable material forming the staples and the resected tissue sample is tested to determine the presence of a disease in the resected tissue sample.

### BRIEF DESCRIPTION OF THE DRAWINGS

Various aspects of the disclosed surgical stapler and methods are described herein below with reference to the drawings, wherein:

FIG. 1 is a side perspective view of a surgical stapling device including a tool assembly having a staple cartridge;

FIG. 2 is a side perspective view of the staple cartridge illustrated in FIG. 1 with staples of the staple cartridge separated from the staple cartridge; and

FIG. 3 is a top view of tissue sections dissected and stapled with the staple cartridge illustrated in FIG. 2.

### DETAILED DESCRIPTION

The disclosed surgical stapler and methods for its use will now be described in detail with reference to the drawings, in which like reference numerals designate identical or corresponding elements in each of the several views. However, it is to be understood that the disclosed aspects of the present disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure. In addition, directional terms such as front, rear, upper, lower, top, bottom, distal, proximal, and similar terms are used to assist in understanding the description and are not intended to limit the disclosure.

The disclosed methods use selectively degradable materials in forming staples for closing wounds in resecting tissue from a patient's body. The staples of the present disclosure are degradable in vivo, or undergo minimal degradation in vivo, but may be subjected to in vitro treatments by a pathologist or similar hospital personnel to accelerate their degradation and permit testing of resected tissue without requiring manual removal of the staples or discarding of tissue including the staples. This allows pathologists better access to stapled resected tissue specimens to improve tissue analysis, including cancer margin analysis.

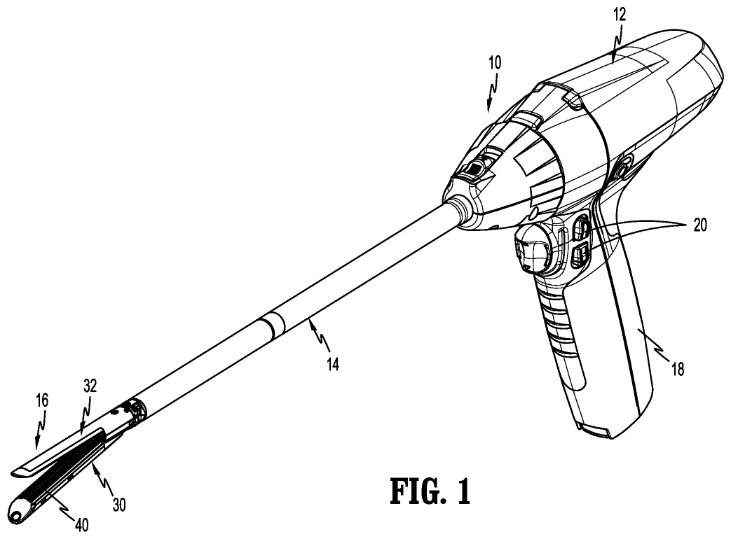

FIG. 1 illustrates a surgical stapling device shown generally as stapling device 10 that includes a handle assembly 12, an elongate body or adapter 14, and a tool assembly 16. In the illustrated aspect, the handle assembly 12 is powered and includes a stationary handgrip 18 and actuation buttons 20. The actuation buttons 20 are operable to actuate various functions of the tool assembly 16 via the adapter 14 including approximation, stapling, and dissection. In aspects of the present disclosure, the handle assembly 12 supports batteries (not shown) that provide energy to the handle assembly 12 to operate the stapling device 10. Although the stapling device 10 is illustrated as a powered stapling device, it is envisioned that the advantages of this disclosure are suitable for use with manually powered surgical stapling devices as well as robotically controlled stapling devices.

The tool assembly 16 of the stapling device 10 includes a cartridge assembly 30 and an anvil 32. As is known in the art, the cartridge assembly 30 and the anvil 32 are coupled together such that the tool assembly 16 can pivot between an open position and a clamped position. The cartridge assembly 30 includes an exemplary aspect of the disclosed staple cartridge shown generally as staple cartridge 40.

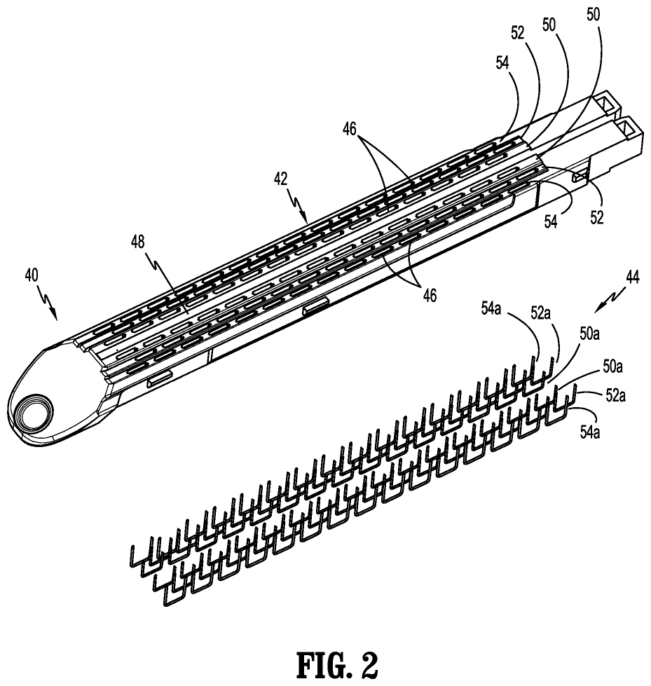

FIG. 2 illustrates the staple cartridge 40 which includes a cartridge body 42 and a plurality of staples 44. The cartridge body 42 defines a plurality of staple pockets 46 and a central knife slot 48 that extends along a midline of the cartridge body 42. As known in the art, the central knife slot 48 facilitates translation of a knife bar (not shown) through the tool assembly 16 to eject staples supported within a staple cartridge and cut tissue clamped between the cartridge assembly 30 and anvil 32. U.S. Pat. No. 5,865,361 discloses a manually powered stapling device including a knife bar that is movable through the tool assembly to eject staples from a staple cartridge and to cut tissue clamped between an anvil and a cartridge assembly of the stapling device. Although the cartridge body 42 is illustrated as being linear, it is envisioned that the cartridge body 42 may have a non-linear configuration or curved along its longitudinal axis.

It is envisioned that the staple pockets 46 in the cartridge body 44 need not be arranged in rows as illustrated above but rather may be arranged in a variety of different patterns in the cartridge body.

In aspects of the present disclosure, the staple pockets 46 are arranged in rows 50, 52, and 54 that are positioned on each side of the central knife slot 48 in the staple pockets 46 of the cartridge body 42. Each of the staple pockets 46 supports a staple 44 such that the staples 44 are aligned in rows 50a, 52a, and 54a on each side of the central knife slot 48 within the cartridge body 42 of the staple cartridge 40. It is noted that the rows need not be linear but rather may be curved along the longitudinal axis of the cartridge body 42. The inner rows 50a of staples 44 are positioned closest to and on opposite sides of the central knife slot 48. The middle and outer rows 52a and 54a of the staples 44 are positioned outwardly of the central knife slot 48 and of the inner rows 50a of staples 44 on opposite sides of the central knife slot 48. Although the staple cartridge 40 is illustrated to include three rows of staples 44 on each side of the central knife slot 48, it is envisioned that the staple cartridge 40 may include only two rows of staples 44 or four or more rows of staples 44 on each side of the knife slot 48.

Staples 44 are formed of material(s) which will not break down or break down slowly to permit healing within the patient. However, the staples 44 can quickly degrade when subjected to a specific treatment by a pathologist, for example exposure to an acidic solution. Staples 44 in the reload are all made of this material(s) and thus there is no specific designated left and right specimen orientation. Thus, no specific orientation of the stapler is required in stapling tissue.

Materials for forming staples 44 include, for example, magnesium, zinc, combinations thereof, and their alloys. Suitable magnesium alloys may include magnesium in combination with zinc and/or silver, for example. Staples including alloys of magnesium and/or zinc increase the healing performance of tissue. Staples including silver provide anti-microbial benefits. Staples including all three alloys may possess both enhanced healing and anti-microbial benefits.

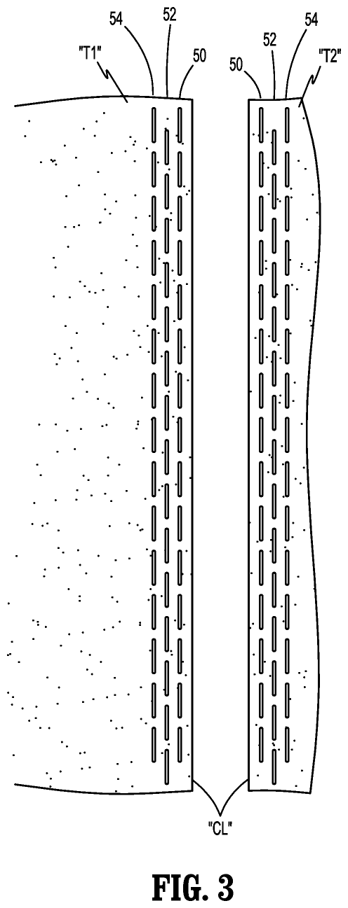

FIG. 3 illustrates dissected tissue sections “T1” and “T2” including staples 44 formed in three rows 50, 52, and 54 along a cut line “CL” in each tissue section “T1” and “T2”. As depicted, “T1” is the tissue remaining within the patient, while “T2” is the resected tissue specimen that is sent to the histopathology department for testing to determine the presence of a disease, such as cancer.

In other aspects of the present disclosure, not all rows of staples 44 are degradable. For example, the 2 inner rows 50a, 52a closest to the knife slot 48 can be degradable while the outer row 54a can be formed of a material not formed of a degradable material, for example titanium, to ensure sealing. Other configurations, including combinations of degradable staples and non-degradable staples, in aspects alternating configurations, are also contemplated.

Once stapling is complete, the resected tissue “T2” is sent to the pathology department where the staples are subjected to accelerated degradation. For example, the resected tissue “T2” may be placed in a staple dissolving solution (not shown). Suitable solutions may include acids, bases, salts, etc. In aspects, a staple dissolving solution may include an acid such as hydrochloric acid, sulfuric acid, acetic acid, nitric acid, combinations thereof, and the like, or salt solutions, which produce high chloride concentrations, such as potassium chloride, sodium chloride, calcium chloride, or the like. Chloride containing salts may also be combined with acids to further accelerate staple dissolution.

Such solutions may be at a concentration sufficient to dissolve the staple, but not harm the resected tissue “T2” to be analyzed. Acid solution concentrations depend on the acid in the solution, and can include, for example: hydrochloric acid at a concentration from about 0.05 N to about 12.07 N, in embodiments from about 0.1 N to about 6 N; acetic acid at a concentration from about 0.05 N to about 17.4 N, in embodiments from about 0.1 N to about 17.4 N; sulfuric acid at a concentration from about 0.05 N to about 36 N, in embodiments from about 0.1 N to about 4 N; nitric acid at a concentration from about 0.05 N to about 24 N, in embodiments from about 0.1 N to about 16 N.

Isotonic salt solutions (0.154 M NaCl for example) may be used in aspects to minimize changes to tissue over time, but short exposure to greater concentrations such as greater than 0.154 M could be feasible to more rapidly dissolve the staples without causing artifacts in the tissue.

Depending upon the concentration of the solution and the degradable material used, the time for degradation of the staples 44 may be from 2 seconds to 10 minutes, in aspects from about 10 seconds to about 5 minutes, in other aspects from about 15 seconds to about 2 minutes. For example, where a magnesium alloy is used, the acid may degrade the staple in a time from about 2 seconds to about 10 minutes.

In other aspects, where intra-operative histology is not occurring, the concentration of the solution may be low, permitting dissolution of the staples over an extended period of time, for example, overnight.

In some aspects, the tissue is rinsed in an appropriate fluid to neutralize the acid and/or remove the acid, and make the process safer for the pathologist. Rinsing the tissue may also be desirable if the tissue is to be preserved for a longer period of time.

Once all the staples 44 have been dissolved from the resected tissue “T2”, the pathologist can perform their normal duties on the resected tissue “T2”. The solution used to degrade the staple may result in some discoloration of resected tissue “T2” to be examined, but there is no visual loss of mass of the tissue. There is no major damage to the resected tissue “T2” as a result of the acid treatment, so the pathologist can proceed to analyze the tissue for disease states, including cancer.

Although only magnesium, magnesium alloys, and titanium have been specifically identified herein as materials suitable for use, it is envisioned that a variety of similar materials could be used to form staples, including zinc, copper, silver, combinations thereof, and the like.

Advantages of the present disclosure include, but are not limited to:
Quick and easy way to remove staples from resected tissue for analysis by pathologists, including analysis of cancer margins; and
Does not require reload having a fixed orientation for use and need for additional labeling.

Persons skilled in the art will understand that the devices and methods specifically described herein and illustrated in the accompanying drawings are non-limiting exemplary aspects of the present disclosure. It is envisioned that the elements and features illustrated or described in connection with one exemplary aspect may be combined with the elements and features of another without departing from the scope of the disclosure. As well, one skilled in the art will appreciate further features and advantages of the disclosure based on the above-described aspects. Accordingly, the disclosure is not to be limited by what has been particularly shown and described, except as indicated by the appended claims.

What is claimed is:

1. A method comprising:
introducing to a patient a surgical stapler, the stapler including staples formed of a degradable material;
using the stapler to staple tissue adjacent an undesired portion of an organ and cutting the tissue to obtain a resected tissue sample;
removing the resected tissue sample from the patient; and
contacting the staples with a solution to degrade the degradable material forming the staples.

2. The method of claim 1, further comprising testing the resected tissue sample to determine the presence of a disease in the resected tissue sample.

3. The method of claim 2, wherein testing the resected tissue sample is conducted to determine the presence of cancer in the resected tissue sample.

4. The method of claim 1, wherein the staples are formed of magnesium or zinc.

5. The method of claim 4, wherein the staples are formed of a magnesium alloy or a zinc alloy.

6. The method of claim 1, wherein the solution to degrade the degradable material forming the staples includes an acid.

7. The method of claim 6, wherein the acid is selected from hydrochloric acid, sulfuric acid, acetic acid, nitric acid, or combinations thereof.

8. The method of claim 1, wherein the solution to degrade the degradable material forming the staples includes a chloride salt.

9. A method comprising:
introducing to a patient a surgical stapler, the stapler including staples formed of a degradable material;
using the stapler to staple tissue adjacent an undesired portion of an organ and cutting the tissue to obtain a resected tissue sample;
removing the resected tissue sample from the patient;
contacting the staples with a solution to degrade the degradable material forming the staples; and
testing the resected tissue sample to determine the presence of a disease in the resected tissue sample.

10. The method of claim 9, wherein testing the resected tissue sample is conducted to determine the presence of cancer in the resected tissue sample.

11. The method of claim 9, wherein the staples are formed of magnesium or zinc.

12. The method of claim 11, wherein the staples are formed of a magnesium alloy or a zinc alloy.

13. The method of claim 9, wherein the solution to degrade the degradable material forming the staples includes an acid.

14. The method of claim 13, wherein the acid is selected from hydrochloric acid, sulfuric acid, acetic acid, nitric acid, or combinations thereof.

15. The method of claim 9, wherein the solution to degrade the degradable material forming the staples includes a chloride salt.
